(19) United States
(12) Patent Application Publication
(10) Pub. No.: US 2004/0064047 A1
(43) Pub. Date: Apr. 1, 2004

(54) Optimization of excitation waveform for nonlinear transmit-receive systems

(75) Inventors: Vasilis Z. Marmarelis, Irvine, CA (US); Tae-Seong Kim, Fullerton, CA (US)

Correspondence Address: MCDERMOTT, WILL & EMERY, Suite 3400, 2049 Century Park East, Los Angeles, CA, 90067, US

(73) Assignees: Alfred E. Mann Institute for Biomedical Engineering at the University of; Southern California

(21) Appl. No.: 10/623,426

(22) Filed: Jul. 18, 2003

Related U.S. Application Data
(60) Provisional application No. 60/397,378, filed on Jul. 19, 2002.

(51) Int. Cl. A61B 8/00

(52) U.S. Cl. 600/437

(57) ABSTRACT

A process for optimizing the excitation waveform that is delivered to a transmitter that, together with a receiver, form part of a nonlinear transmission and reception system. The process may include delivering a transmission test signal to the transmitter, generating a receive test signal from the receiver that is a nonlinear function of the transmission test signal, developing a nonlinear model of the nonlinear function, and determining an optimum excitation signal based on the model. An application to ultrasonic imaging for breast mammography is also disclosed.

### CROSS-REFERENCE TO RELATED APPLICATIONS

This application is based upon and claims priority to U.S. Provisional Application Serial No. 60/397,378, entitled “Optimization of the Excitation Waveform for Transmit-Receive Systems,” filed Jul. 19, 2002, the entire content of which is incorporated herein by reference.

### BACKGROUND OF INVENTION

1. Field of Invention

This invention relates to transmit-receive systems and, more particularly, to optimizing the excitation waveform that is used in such systems to maximize the output for a given constraint on the input.

2. Description of Related Art

Systems employing a transmitter and receiver are widely used. Applications include telemetry, imaging and communications. A specific example of an imaging application is ultrasonic transmission-mode tomography used in breast mammography.

A signal of a selected waveform is typically delivered to the transmitter, emitted by the transmitter, propagated though a medium, and received at the receiver. Information of interest is then extracted.

To maximize the signal-to-noise ratio, it is often desirable to maximize the output at the receiver based on a given constraint on the input to the transmitter.

One approach has been to optimize the design of the individual hardware components, such as the transducers and electronics. For example, the thickness and impedance of the various matching layers in the transducers have been adjusted. Appropriate tuning circuitry has also been introduced to maximize the effect of the excitation waveform on the transmitted transducer.

These types of tuning methods can be problematic. The impedance of the electronic circuits associated with the transmitter and receiver may be different, requiring different tuning conditions during transmission and reception. Precise knowledge of the dynamic characteristics of the transducers may also be needed in conjunction with the associated electronics.

Rather than physically tuning system components, transmit-receive systems have been modeled as linear systems. Based on this modeling, the center frequency, shape, duration and amplitude of the excitation waveform has been adjusted to maximize the peak received value predicted by these models.

Unfortunately, there are often inherent nonlinearities in transmit-receive systems, including in ultrasonic transmit-receive systems, that can cause the results of this linear modeling approach to be less than optimal. In turn, the greatest possible signal-to-noise ratio may not be achieved.

### SUMMARY OF INVENTION

A process for optimizing the excitation waveform that is delivered to an ultrasonic transmitter that, together with an ultrasonic receiver, form part of a nonlinear ultrasonic transmission and reception system. A transmission test signal may be delivered to the ultrasonic transmitter. A received test signal may be generated from the ultrasonic receiver that is a nonlinear function of the transmission test signal. A nonlinear model of the nonlinear function may be developed from the transmission test signal and the received test signal. An optimum excitation signal for the ultrasonic transmitter may be determined that substantially maximizes the signal generated by the ultrasonic receiver based on the model and based on a specified constraint on the excitation signal.

The kernel functions of the nonlinear function may be determined as part of the process of developing the model. An algorithm may be used in determining the kernel functions. Principal dynamic modes of the nonlinear function may also be determined as part of this process and may be based on the kernel functions.

The time inversion of one or more of the principal dynamic modes may be determined as part of determining the optimum excitation signal.

One or more of the kernel functions may be excluded when calculating the time inversion.

A Laguerre-Volterra network may be used in developing the nonlinear model. Parameters of the Laguerre-Volterra network may be adjusted to minimize the mean-squared error between the signal predicted by the network and the received test signal. The adjustment may be an iterative process.

The nonlinear model may include a linear filter followed by a static nonlinearity.

The specified constraint on the excitation signal may include a constraint on the amplitude or power of the excitation signal.

The amplitude or power of the signal generated by the ultrasonic receiver may be maximized as part of determining the optimum excitation signal.

The transmission test signal may be a wideband signal. The wideband signal may cover the bandwidth over which the ultrasonic transmitter is configured to operate. The wideband signal may be white noise or a chirp.

The transmission test signal may cover the dynamic range over which the ultrasonic transmitter is configured to operate.

The ultrasonic transmitter may be excited with the optimum excitation signal. A breast may be placed between the ultrasonic transmitter and the ultrasonic receiver during this process. The signal received by the ultrasonic receiver in response to the optimum excitation signal may be analyzed to create an image of the breast.

An ultrasonic imaging system for generating an image of tissue may include an ultrasonic transmitter that converts an excitation signal into an ultrasonic signal; an ultrasonic receiver positioned to receive the ultrasonic signal transmitted by the ultrasonic transmitter and that generates a received signal that is a nonlinear function of the excitation signal; an excitation signal generator in communication with the ultrasonic transmitter that generates an excitation signal that substantially maximizes the signal generated by the ultrasonic receiver based on a specified constraint on the excitation signal; and a processing system in communication with the ultrasonic receiver for processing the signal generated by the ultrasonic receiver into an image of tissue disposed between the ultrasonic transmitter and the ultrasonic receiver.

The signal generated by the excitation signal generator may be derived from a nonlinear model of the nonlinear function. The nonlinear model may be developed from a comparison of a transmitted test signal transmitted by the ultrasonic transmitter and a received test signal generated by the ultrasonic receiver.

These processes and system may also be used in transmit-receive systems that operate outside of the ultrasonic range.

These as well as still further features, benefits and objects will now become clear upon an examination of the following detailed description of illustrative embodiments and the attached drawings.

### BRIEF DESCRIPTION OF DRAWINGS

FIG. 1 is a block diagram of a transmit-receive system.

FIG. 2 is a block diagram of a process for determining the optimum excitation signal for a transmitter in a non-linear transmit-receive system.

FIG. 3 is a diagram of a Laguerre Volterra Network.

FIG. 4 is a diagram of parallel cascades of linear filters and their associated nonlinearities.

FIG. 5 is non-linear model of a transmit-receive system using a linear filter and a static nonlinearity.

FIGS. 6(a)-6(d) are traces of measured and predicted signals in a non-linear transmit-receive system.

FIG. 7 is a block diagram of a non-linear model of a non-linear system using principal dynamic modes.

FIGS. 8(a)-(d) are traces of optimal and pulse excitation signals and their corresponding received signals.

### DETAILED DESCRIPTION OF ILLUSTRATIVE EMBODIMENTS

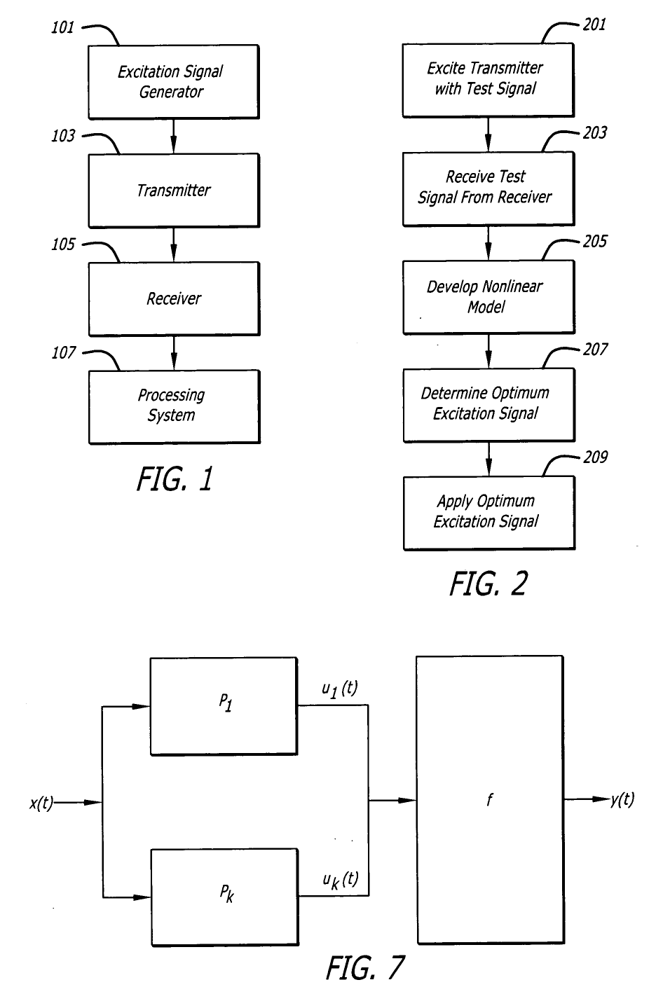

FIG. 1 is a block diagram of a transmit-receive system.

As shown in FIG. 1, an excitation signal generator 101 may generate an excitation signal. This signal may be a steady-state signal, a series of pulses, or any other type of signal.

The signal generated by the excitation signal generator 101 may be delivered to a transmitter 103. The transmitter 103 may be an ultrasonic transmitter or any other type of transmitter. The transmitter may be a single transmitter or an array of transmitters. In the case of an array of transmitters, separate signals may be delivered to each element in the array from the excitation signal generator 101. The separate signals may be the same or different.

The signal transmitted by the transmitter 103 may travel through a propagation medium, such a breast suspended within acoustical coupling fluid, and then arrive at a receiver 105. The receiver 105 may be an ultrasonic receiver or any other type of receiver. As with the transmitter 103, the receiver 105 may be a single receiver or an array of receivers.

The signal generated by the ultrasonic receiver 105 (or signals in the case of an array) may be directed to a processing system 107. The processing system may analyze the received signal, possibly along with the excitation signal, to extract information that is of interest. In one embodiment, the information of interest may be a three-dimensional image of a breast interposed between the transmitter 103 and the receiver 105. More details concerning transmission mode tomographic imaging for breast mammography are set forth in U.S. patent application Ser. No. 10/117,763, filed Apr. 5, 2002, entitled “High Resolution 3D Ultrasonic Transmission Imaging,” U.S. patent application Ser. No. 10/117,468, filed Apr. 5, 2002, entitled “Nonlinear Processing for Mitigation of Diffraction Effects,” U.S. Provisional Application Serial No. 60/396,516, filed Jul. 16, 2002, entitled “Support Bra for Ultrasonic Breast Scanner,” U.S. patent application Ser. No. 10/382,235, filed Mar. 5, 2003, entitled “Multi-Mode Processing for Ultrasonic Imaging,” U.S. Provisional Application Serial No. 60/478,981, filed Jun. 17, 2003, entitled “Targeted Ultrasonic Hyperthermia With A High-Resolution 3-D Fixed Coordinate System,” and U.S. patent application filed Jun. 27, 2003, entitled “Three-Dimensional Ultrasonic Mammography Scanning,” attorney docket number 64693-070. The entire content of all of these applications is incorporated herein by reference.

FIG. 2 is a block diagram of a process for determining the optimum excitation signal for a transmitter in a nonlinear transmit-receive system.

The transmitter 103 may be excited with a test signal, as reflected by an Excite Transmitter With Test Signal step 201. The test signal may be a wideband signal. It may be bandwidth-limited white noise, such as white noise with a bandwidth of 4-24 MHz. It may be a chirp. The wideband test signal may be selected to cover the bandwidth and dynamic range over which the transmitter 103, the receiver 105 and their associated electronics are configured to operate during normal use. The test signal may be any other form of signal.

The test signal may travel through the anticipated propagation medium, such as acoustic coupling fluid containing a breast dangling therein. The test signal may then reach the receiver 105. The receiver 105 may then generate a received test signal, as reflected by a Receive Test Signal from Receiver step 203.

The excitation signal and the signal generated by the receiver may be stored for later analysis. If desired, the signals may be digitized either before or after their storage. A time-bandwidth product of approximately 1,000 may be used for this purpose which, in the case of an excitation signal having a bandwidth of approximately 4-24 Megahertz, may be approximately 100 MHz.

The signal generated by the receiver 105 may be a nonlinear function of the excitation signal generated by the excitation signal generator 101. The nonlinearity may be caused by nonlinearity in the transmitter 103, the receiver 105, the excitation signal generator 101, the propagation medium, and/or by other areas of the system.

A nonlinear model of the dynamic transformation between the excitation signal and the signal generated by the receiver may be developed, as reflected by a Develop Nonlinear Model step 205. This may be a mathematical model that is derived using any type of nonlinear system identification method.

Based on the nonlinear model that is developed, an optimum excitation signal may be determined that optimizes the signal generated by the receiver 105 based on a constraint on the excitation signal that is delivered to the transmitter 103. Any technique for determining the optimum excitation signal based on the nonlinear model may be used.

The constraint on the excitation signal may include a constraint on its peak amplitude, power and/or other characteristics.

The excitation signal may be optimized to maximize the amplitude, power and/or other characteristics in the signal generated by the receiver 105.

The optimum excitation signal may then be applied to the transmitter 103, as reflected by an Apply Optimum Excitation Signal step 209. To this end, the excitation signal generator 101 may be configured to generate the optimum excitation signal during the normal operation of the transmit-receive system.

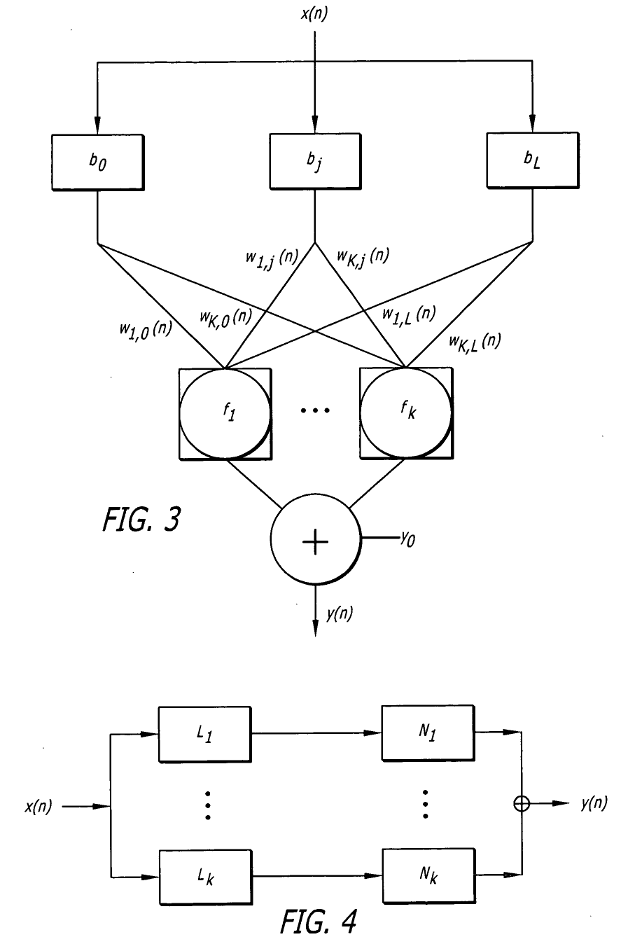

FIG. 3 is a diagram of a Laguerre-Volterra network (LVN). This is an example of a nonlinear system identification method that may be used as part of the process of developing a nonlinear model of the transmit-receive system.

In FIG. 3, x(n) represents the excitation signal generated by the excitation signal generator 101 and y(n) represents a prediction of the signal that is generated by the receiver 105 as a result.

The LVN method may combine expansions on the discrete-time Laguerre basis with feed-forward artificial neural networks (FANN) using polynomial activation functions. It may follow the standard architecture of a single-layer, fully connected FANN with three distinctive features:

(1) The Laguerre-filter bank bⱼ (j=0, 1, . . . , L) that preprocesses the input, x(n);

(2) The polynomial activation functions, fₖ, (k=1, 2, . . . , K) in the hidden units; and

(3) The non-weighted summative output unit.

The architectural parameters of the LVN may consist of the number of Laguerre filters (LFs), hidden units (HUs) and the degree of the polynomial activation functions. The selection of these structural parameter values can be performed by successive trials in ascending order, moving from lower to higher numbers, using the normalized mean-squared error (NMSE) of the output prediction achieved by the model as a criterion.

The determination of the optimal number of LFs for a specific system can be assisted by observing the relative magnitude of the Laguerre expansion coefficients. Likewise, the determination of the optimal number of HUs can be assisted by observing the relative magnitudes of the polynomial coefficients, when the inbound weights for each HU are normalized to a unity sum of squares.

Once the LVN architecture is set, the training of the LVN may be performed with input-output experimental data to estimate the values of the unknown network parameters, including the weights wₖⱼ, the polynomial coefficients c_{q,k}, the Laguerre parameter a and the offset y₀. The network training may involve minimizing the mean-square error between the measured output and the LVN predicted output y(n) for the corresponding input. For effective training, the input-output data may be broadband and cover the entire bandwidth and dynamic range of the system.

The output of the j-th discrete-time Laguerre filter with impulse response function bⱼ(m) may be given by the discrete convolution:

$$\begin{matrix}{v}_{j} ( n ) = {\sum }_{m = 0}^{∞} {b}_{j} ( m ) x ( n - m ) \\ where & ( 1 ) \\ {b}_{j} ( m ) = {{a}^{( m - j ) / 2} ( 1 - a )}^{1 / 2} {\sum }_{i = 0}^{j} {( - 1 )}^{i} ( \begin{matrix}m \\ i\end{matrix} ) ( \begin{matrix}j \\ i\end{matrix} ) {{a}^{j - 1} ( 1 - a )}^{i} & ( 2 )\end{matrix}$$

and a is the discrete-time Laguerre parameter.

The input of the k-th hidden unit may be a weighted sum of the Laguerre filter bank outputs:

$${u}_{k} ( n ) = {\sum }_{j = 0}^{L} {w}_{k , j} {v}_{j} ( n ) ( k = 1 , 2 … K ) \qquad ( 3 )$$

The corresponding output of the k-th hidden unit may be given by the polynomial activation function:

$${z}_{k} ( n ) = {\sum }_{q = 0}^{Q} {c}_{q , k} {u}_{k}^{q} ( n ) ( k = 1 , 2 … K ) \qquad ( 4 )$$

The network output may be given by a non-weighted summation of the hidden unit outputs including a trainable offset value, y₀:

$$y ( n ) = {\sum }_{k = 1}^{K} {Z}_{k} ( n ) + {y}_{0} \qquad ( 5 )$$

The training of the network parameters may be performed iteratively using a gradient descent method for a quadratic cost function defined by the square of the output prediction error.

As shown in FIG. 4, the LVN architecture shown in FIG. 3 may generally be expressed as K parallel cascades of linear filters L₁ . . . Lₖ and their associated static nonlinearities N₁ . . . Nₖ. FIG. 4 shows the equivalent version of the LVN where the input x(n) goes through a set of K linear filters (determined by the weights wₖⱼ) and their associated nonlinearities (determined by the polynomial coefficients c_{q,k}). The output may be formed by summing all of the outputs of the static nonlinearities.

More details concerning the use of a Laguerre-Volterra network as a nonlinear system identification method are set forth in V. Z. Marmarelis, “Modeling Methodology for Nonlinear Physiological Systems,” Annals of Biomedical Engineering, Vol. 25, pp. 239-251 (1997), the entire content of which is incorporated herein by reference.

Once the network parameters are found, the corresponding model of kernel functions may be constructed in accordance with well known techniques. The obtained mathematical model may describe the nonlinear dynamic transformation of the input excitation signal into the output signal generated by the receiver in the transmit-receive system.

In one experiment, the test signal was a wideband chirp covering a bandwidth from approximately 4 to 20 MHz over a dynamic range of ±50 volts. The received signal was recorded over 5,000 samples at 20 nsec sampling intervals.

The excitation waveform was generated from an arbitrary waveform generator, such as an HP 33250A from Hewlett-Packard in Palo Alto, Calif., with a pre-designed wideband waveform covering this bandwidth and dynamic range. The system output was measured at the receiving transducer and digitized using an oscilloscope, such as the TDS5054 Digital Phosphor Oscilloscope from Tektronix in Beaverton, Oreg.

The digitized transmitted and received signals were analyzed using the LVN nonlinear system identification method described above to obtain a nonlinear model of the dynamic transformation of this transmit-receive system for any excitation waveform into the corresponding received waveform.

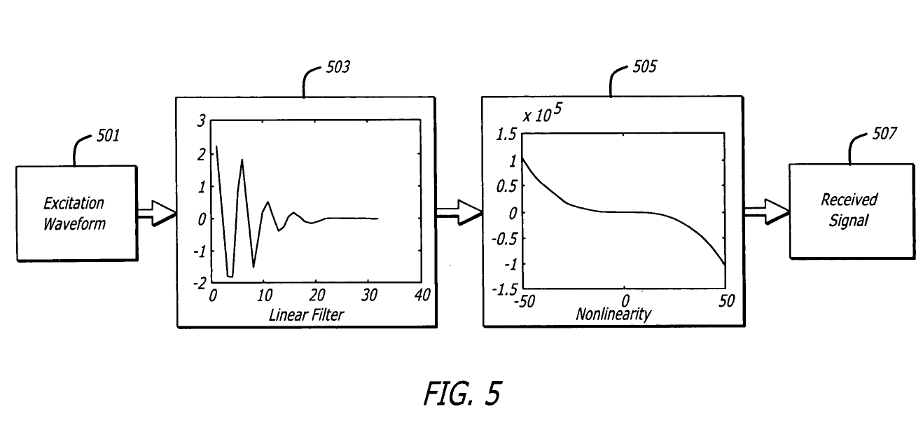

Application of the LVN modeling methodology to the collected data yields the nonlinear model shown in FIG. 5. This is comprised of the excitation waveform 501 being delivered to a linear filter 503, followed by a cubic static nonlinearity 505 and a received signal 507.

With the LVN structural parameters of 17 Laguerre functions, 2 hidden units, and a third-degree polynomial activation function, the normalized mean squared-error of the model prediction with respect to the measured output data was 5.9% after 100 iterations. In all trials with different structural parameters, only one hidden unit was found to be active. This allowed the model to be simplified to the single cascade shown in FIG. 5.

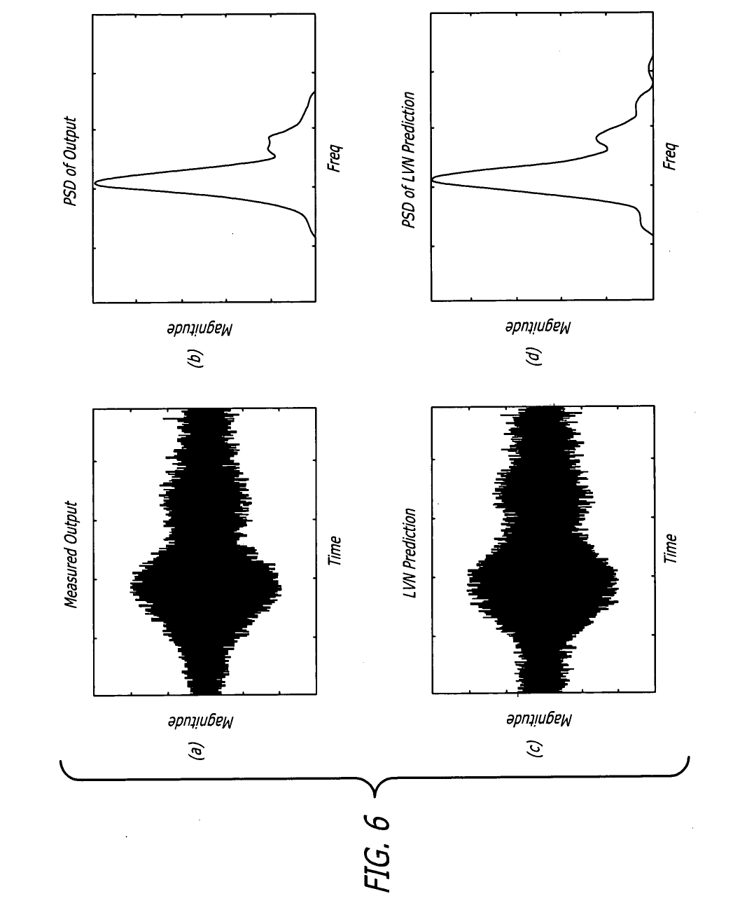

FIG. 6 shows the measured receiver output and the LVN model predictions along with their power spectra. The accuracy of the LVN model prediction is evident in both the time and the frequency domains.

The optimal excitation waveform may be derived from this model on the basis of the matched-filter principle, that is, by determining the time-reversed impulse response of the linear filter of the cascade model, scaled according to an input power constraint. The optimal excitation pulse may match the frequency response of the linear filter. The static nonlinearity may determine the final gain of the output and indicate that doubling of the input more than doubles the output (supralinear relation).

FIG. 7 is a block diagram of a nonlinear model of a nonlinear system using principal dynamic modes. The P₁ . . . Pₖ blocks represent the principal dynamic modes (PDM) of the system based on the kernel functions. The u₁(t) . . . uₖ(t) represent a convolution of the input x(t) with each of the principal dynamic modes. The f block represents the static point-to-point mapping.

Mathematically, y(t) in FIG. 7 may be expressed as:

y(t)=f{u₁(t), . . . , uₖ(t)} (6)

The input signal x(t) may be expressed as:

$$x ( t ) = {\sum }_{i = 1}^{K} {λ}_{i} {P}_{i} ( - t ) \qquad ( 7 )$$

where λᵢ are scalers.

By applying the input signal, uⱼ(t) may be expressed as:

$$\begin{matrix}{u}_{j} ( t ) = {\sum }_{i = 1}^{K} {λ}_{i} {\int }_{0}^{P} {p}_{j} ( τ ) {p}_{i} ( τ - t ) ⅆ τ = & ( 8 ) \\ {\sum }_{i = 1}^{K} {λ}_{i} {Z}_{j , i} ( t ) & ( 9 )\end{matrix}$$

and y(t) may be expressed as:

y(t)=f{λᵢ, . . . , λ_{K}} (10)

where Z_{j,i} (t) are known.

y(t) may then be optimized over the parameters {λ₁, . . . ,λₖ}subject to a constraint:

$${\sum }_{i} ( {λ}_{1}^{*} , … , {λ}_{k}^{*} ) \qquad ( 11 )$$

For example, the values (λ₁, . . . , λ_{K}) may be determined for the maximum value of y(t) over t is greatest subject to the constraint:

$${\sum }_{i} {( {λ}_{1}^{*} )}^{2} = constant \qquad ( 12 )$$

Other optimization criteria, such as maximizing the power of y(t) and/or constraints such as 0<λᵢ<1, can also be applied.

As indicated, these are examples of approaches for determining an optimum excitation signal based on an input constraint.

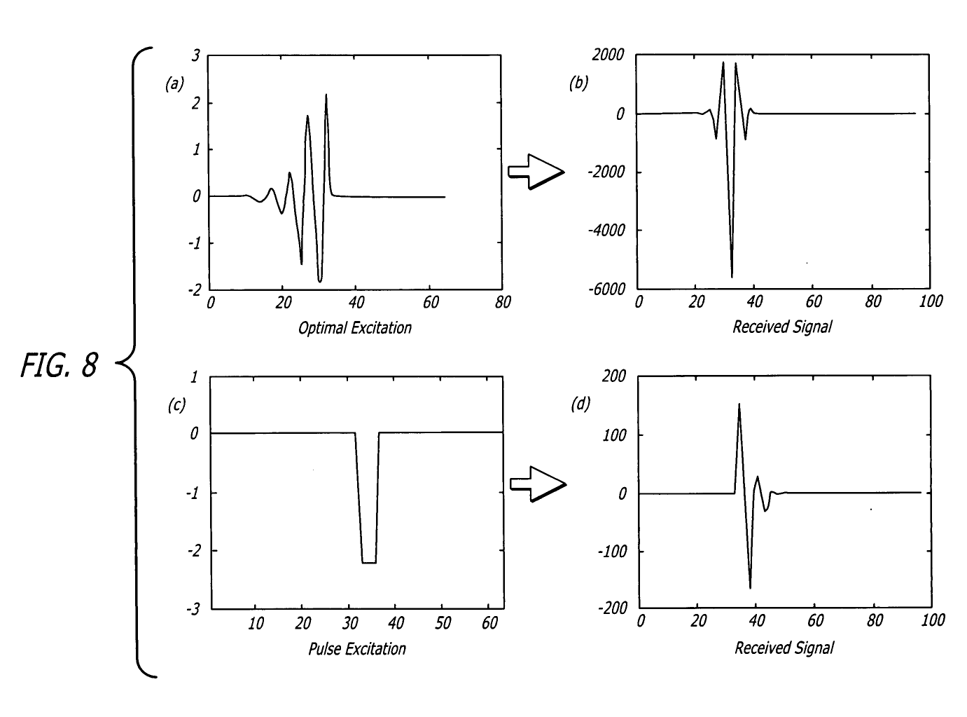

FIGS. 8(a)-(d) are traces of optimal and pulse excitation signals and their corresponding received signals. FIG. 8(a) is a trace of the optimal excitation signal that was determined based on the principals discussed above; FIG. 8(b) is a trace of the signal generated by the receiver of this optimal excitation signal; FIG. 8(c) is a trace of a non-optimal pulse that was commonly used to excite a transmitter; and FIG. 8(d) is a trace of the signal generated by the receiver of this non-optimal pulse signal. As can be seen by a comparison of the traces in FIGS. 8(b) and (d), use of the optimal excitation signal increased the amplitude of the received signal by about 35 times, without any significant increase in the amplitude of the input signal.

Although a test signal has been described as a mechanism for obtaining the nonlinear model of the system, other approaches could be used instead. In short, what has thus-far been described are merely examples of the components, steps, features and benefits of the invention. The invention is not limited to these, but solely to the subject matter delineated by the following claims and their equivalents.

We claim:

1. A process for optimizing the excitation waveform that is delivered to an ultrasonic transmitter that, together with an ultrasonic receiver, form part of a nonlinear ultrasonic transmission and reception system comprising:
delivering a transmission test signal to the ultrasonic transmitter;
generating a received test signal from the ultrasonic receiver that is a nonlinear function of the transmission test signal;
developing a nonlinear model of the nonlinear function from the transmission test signal and the received test signal; and
determining an optimum excitation signal for the ultrasonic transmitter that substantially maximizes the signal generated by the ultrasonic receiver based on the model and based on a specified constraint on the excitation signal.

2. The process of claim 1 wherein developing the nonlinear model includes determining kernel functions of the nonlinear function.

3. The process of claim 2 wherein an algorithm is used in determining the kernel functions.

4. The process of claim 2 wherein developing the nonlinear model includes determining principal dynamic modes of the nonlinear function based on the kernel functions.

5. The process of claim 4 wherein determining an optimum excitation signal includes calculating the time inversion of one or more of the principal dynamic modes.

6. The process of claim 5 wherein one or more of the kernel functions are excluded when calculating the time inversion.

7. The process of claim 1 wherein developing the nonlinear model includes determining principal dynamic modes of the nonlinear function.

8. The process of claim 1 wherein a Laguerre-Volterra network is used in developing the nonlinear model.

9. The process of claim 8 wherein parameters of the Laguerre-Volterra network are adjusted to minimize the mean-squared error between the signal predicted by the network and the received test signal.

10. The process of claim 9 wherein the adjustment is an iterative process.

11. The process of claim 1 wherein the nonlinear model includes a linear filter followed by a static nonlinearity.

12. The process of claim 1 wherein the specified constraint on the excitation signal includes a constraint on the amplitude of the excitation signal.

13. The process of claim 1 wherein the specified constraint on the excitation signal includes a constraint on the power of the excitation signal

14. The process of claim 1 wherein determining an optimum excitation signal maximizes the amplitude of the signal generated by the ultrasonic receiver.

15. The process of claim 1 wherein determining an optimum excitation maximizes the power of the signal generated by the ultrasonic receiver.

16. The process of claim 1 wherein the transmission test signal is a wideband signal.

17. The process of claim 16 wherein the wideband signal covers the bandwidth over which the transmission and reception system is configured to operate.

18. The process of claim 16 wherein the wideband signal is white noise.

19. The process of claim 16 wherein the wideband signal is a chirp.

20. The process of claim 1 wherein the transmission test signal covers a dynamic range.

21. The process of claim 20 wherein the dynamic range is the dynamic range over which the transmission and reception system is configured to operate.

22. The process of claim 1 further comprising exciting the ultrasonic transmitter with the optimum excitation signal.

23. The process of claim 22 wherein a breast is placed between the ultrasonic transmitter and the ultrasonic receiver while exciting the ultrasonic transmitter with the optimum excitation signal.

24. The process of claim 23 wherein the signal received by the ultrasonic receiver in response to the optimum excitation signal is analyzed to create an image of the breast.

25. An ultrasonic imaging system for generating an image of tissue comprising:
an ultrasonic transmitter that converts an excitation signal into an ultrasonic signal;
an ultrasonic receiver positioned to receive the ultrasonic signal transmitted by the ultrasonic transmitter and that generates a received signal that is a nonlinear function of the excitation signal;
an excitation signal generator in communication with the ultrasonic transmitter that generates an excitation signal that substantially maximizes the signal generated by the ultrasonic receiver based on a specified constraint on the excitation signal; and
a processing system in communication with the ultrasonic receiver for processing the signal generated by the ultrasonic receiver into an image of tissue disposed between the ultrasonic transmitter and ultrasonic receiver.

26. The system of claim 25 wherein the signal generated by the excitation signal generator is derived from a nonlinear model of the nonlinear function.

27. The system of claim 26 wherein the nonlinear model is developed from a comparison of a transmitted test signal transmitted by the ultrasonic transmitter and a received test signal generated by the ultrasonic receiver.

28. A process for optimizing the excitation waveform that is delivered to a transmitter that, together with a receiver, form part of a nonlinear transmission and reception system comprising:
delivering a transmission test signal to the transmitter;
generating a received test signal from the receiver that is a nonlinear function of the transmission test signal;
developing a nonlinear model of the nonlinear function from the transmission test signal and the received test signal; and
determining an optimum excitation signal for the transmitter that substantially maximizes the signal generated by the receiver based on the model and based on a specified constraint on the excitation signal.

29. A nonlinear transmission and reception system comprising:
a transmitter that converts an excitation signal into a transmitted signal;
a receiver positioned to receive the transmitted signal and that generates a received signal that is a nonlinear function of the excitation signal; and
an excitation signal generator in communication with the transmitter that generates an excitation signal that substantially maximizes the signal generated by the ultrasonic receiver based on a specified constraint on the excitation signal.

30. The system of claim 29 wherein the signal generated by the excitation signal generator is derived from a nonlinear model of the nonlinear function.

31. The system of claim 30 wherein the nonlinear model is developed from a comparison of a transmitted test signal transmitted by the transmitter and a received test signal generated by the receiver.
